(19) United States
(12) Patent Application Publication
(10) Pub. No.: US 2004/0147929 A1
(43) Pub. Date: Jul. 29, 2004

(54) Tubular element for an implant for use in spine or bone surgery and implant having such an element

(75) Inventors: Lutz Biedermann, Vs-Villingen (DE); Jurgen Harms, Karlsruhe (DE)

Correspondence Address: EDWARDS & ANGELL, LLP, P.O. BOX 55874, BOSTON, MA, 02205, US

(73) Assignee: Biedermann Motech GmbH

(21) Appl. No.: 10/740,868

(22) Filed: Dec. 19, 2003

(30) Foreign Application Priority Data
Dec. 20, 2002 (DE) 102 60 222.0

(51) Int. Cl. A61B 17/56; A61F 2/44

(52) U.S. Cl. 606/61; 623/17.11

(57) ABSTRACT

A tubular element is disclosed that is useful for an implant for spine or bone surgery. The element has a cylindrical wall with and a bone thread section with a bone thread and a plurality of apertures in the wall in the bone thread section. At least one end of the element is structured and arranged to receive a head or a tip, and wherein the apertures are located in the root of the bone thread and comprise dimensions such that the crest of the bone thread is intact. Further, implants are described that include as a component the tubular element as well as methods of use of such implants. These implants have the advantage of serving as a fusion element and simultaneously allowing a smooth insertion into bone without causing additional damage of the bone due to apertures.

### FIELD OF THE INVENTION

The invention relates to a tubular element for an implant to be used in spine or bone surgery, an implant having such an element and methods of use.

### BACKGROUND OF THE INVENTION

A tubular element for an implant to be used in spine or bone surgery is described in International Patent Publication WO 02/38054 A2 wherein the element has a bone thread section with a bone thread and a plurality of apertures in the bone thread section. An implant in form of a bone screw having such an element also is described. In case of a bone fracture, this known bone screw makes it possible to fixedly connect parts of the bone by the bone screw acting as a tension element and simultaneously as a fusion element, wherein the apertures in the wall allow bone material or vessels to grow in.

However, the crests of the helix of the bone thread of the known implant are interrupted at the locations where apertures are formed in the wall. Thereby, teeth are formed which have a cutting effect when the bone screw is screwed into the bone and which can damage the bone. In particular for bones weakened, for example by osteoporosis, the use of the known implant can cause problems at the time of screwing in.

German Patent DE 199 49 285 C2 describes a bone screw that comprises a shaft having a bone thread, a tip and a head, which are integrally formed. The bone screw further comprises a coaxial blind bore extending through the head and the shaft from which a plurality of radial bores extend through the screw wall.

The radial bores are arranged between the flanks of the thread of the bone thread, respectively. The bores serve as a channel for the injection and distribution of bone cement. Therefore, it is not possible for bone material or vessels to grow into the bores.

International Patent Publication WO 02/11630 A1 describes a bone anchoring system comprising a bone screw and a mechanical insert received in a central bore of the bone screw. The bone screw defines a self-tapping, self-boring tip for easy bone insertion as well as through-holes or other openings for receipt of new bone growth.

Thus, it can be appreciated that new and improved tubular elements and implants using such elements continue to be sought that are versatile in application.

### SUMMARY OF THE INVENTION

The present invention provides a tubular element for an implant to be used in spine or bone surgery. The tubular element comprises a cylindrical wall having a bone thread section (5; 102) with a bone thread and a plurality of apertures (9, 9′) in the wall in the bone thread section (5; 102), at least one end of the element (1; 101) being structured and arranged to receive a head or a tip. The apertures are located and arranged in the root (8) of the bone thread and are dimensioned such that the crest (7) of the bone thread is intact. Preferably, the apertures are distributed along the entire axial length of the bone thread section of the element.

Certain preferred embodiments of the tubular element comprise one or more of the following features:

i) at least one aperture is dimensioned such that the largest diameter of the aperture (9) is smaller than the distance between adjacent facing thread flanks (6) at the root (8) of the thread;

ii) at least one aperture is dimensioned such that the largest diameter of the aperture (9′) is larger than the distance between adjacent facing thread flanks (6) at the root (8) of the thread, wherein the aperture (9′) then extends into at least one of the flanks (6);

iii) at least one of the apertures (9, 9′) comprises a countersinking at the outside of the wall;

iv) in each turn of the thread a plurality of equally distanced apertures are provided;

v) at least on one end, a section (10, 11; 105, 106) having an inner thread for screwing in a head (16; 24; 40) or a tip (12, 13) is provided; and/or

vi) the bone thread section (102) is conically shaped.

The invention also provides an implant having a tubular element as described above, wherein a tip (12, 12′, 15) is detachably connected at the end of the tubular element (1; 101).

Certain preferred embodiments of the implant comprise one or more of the following features:

i) a head (16; 19; 24; 40, 41, 47, 47′) is detachably connected to the other end of the tubular element (1; 101);

ii) the head is structured and arranged as a screw head (17; 40);

iii) the head is structured and arranged as a substantially cylindrical threaded element (19);

iv) the head (24; 41; 47; 47′) comprises a receiver member for a rod (100, 100′) connecting several implants;

v) the head (24) and the tubular element (1; 101) are monoaxially connected with each other; and/or

vi) the head (40, 41; 40, 47; 40, 47′) and the tubular element (1; 101) are connected polyaxially with each other.

The invention also provides a method for inserting an implant into bone tissue, the method comprising providing a tubular element of suitable length, fixing a tip portion onto one end of the tubular element, fixing a head portion onto the other end of the tubular element, thereby forming a bone screw, and inserting the bone screw into the bone.

In addition, the invention provides a method for fixing bone fractures, the method comprising providing a tubular element of suitable length, fixing a tip portion onto one end of the tubular element, introducing bone material or an agent into the tubular element, fixing a head portion onto the other end of the tubular element, thereby forming a bone screw, inserting the bone screw into the bone to be fixed, and allowing bone tissue to grow around the bone screw and into the apertures.

The invention further provides a method for stabilizing the spine, the method comprising providing a tubular element of suitable length, fixing a tip portion onto one end of the tubular element, introducing bone material or an agent into the tubular element, fixing a head portion onto the other end of the tubular element, wherein the head portion comprises a receiver member for receiving a rod, thereby forming a bone screw with a receiver member, inserting the bone screw into the spine, fixing the rod in the receiver member, and allowing bone tissue to grow around the bone screw and into the apertures. Additional tubular elements can be provided to form bone screws with a receiver member. The additional bone screws are located in additional sites in the spine and the rod fixed in the respective receiver members of the additional bone screws, thereby stabilizing the spine.

Further features and advantages of the invention are apparent from consideration of the description of embodiments and the Figures.

### BRIEF DESCRIPTION OF THE DRAWINGS

FIG. 1 is an elevational view of a tubular element according to one embodiment of the invention;

FIG. 2 is an enlarged elevational view, partly in section, of a portion of the tubular element of FIG. 1;

FIG. 3 is an enlarged elevational view, partly in section, of a modification of a portion of the tubular element of FIG. 1;

FIG. 4 illustrates alternative embodiments of implants using the tubular element of FIG. 1;

FIG. 5 illustrates a further embodiment of an implant using the tubular element of FIG. 1;

FIGS. 6a) to 6c) illustrate further alternative embodiments of implants using the tubular element of FIG. 1;

FIG. 7 is an elevational view of a tubular element according to a second embodiment of the invention.

FIG. 8 is an enlarged elevational view of a modification of the first and second embodiment.

### DETAILED DESCRIPTION OF THE INVENTION

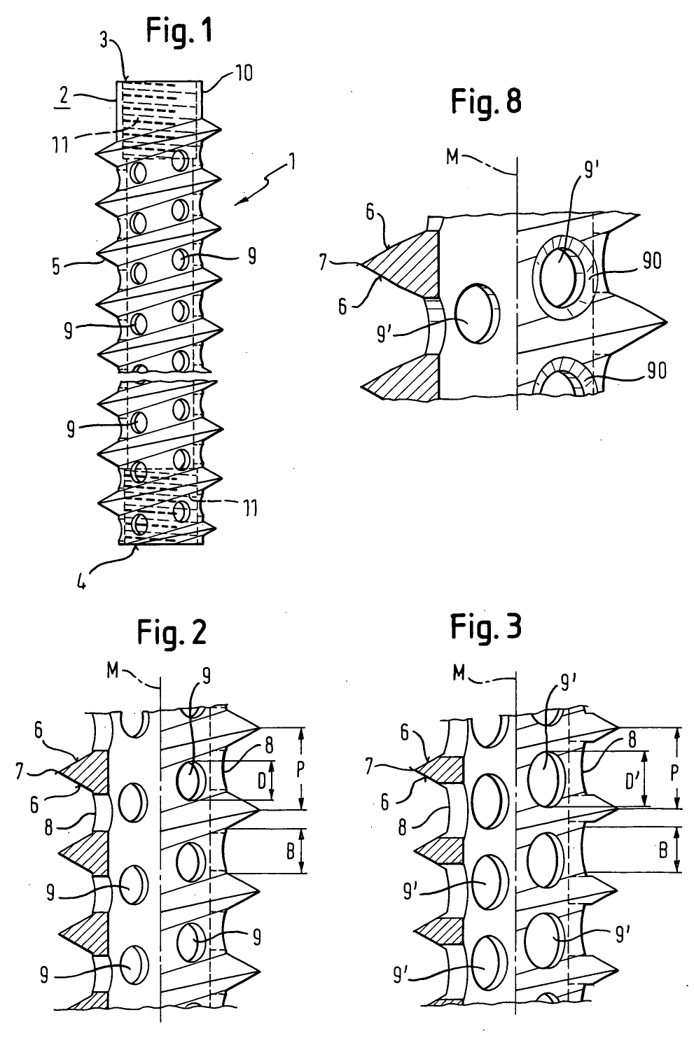

As can be seen best from FIGS. 1 and 2, a tubular element 1 according to a first embodiment of the invention is formed of a cylindrical tube 2, having a first end 3 and a second end 4 opposite to the first end. The tube 2 comprises at its outer wall a bone thread section 5 having a bone thread for screwing the tube into a bone. As illustrated in the drawings, the tube has a wall thickness that is substantially less than the radius of the tube, thereby providing a hollow interior that is a substantial portion of the volume occupied by the element.

Such dimensions can be realized for example as follows: The ratio wdv of the wall thickness of the tubular element to the root diameter of the bone thread is calculated wdv=wd/dk, wherein wd is the wall thickness and dk is the root diameter of the bone thread. wd=(dk−db)/2 wherein db is the diameter of the injection bore. According to the invention the ratio wdv is in the range from around 18% to 25%, preferably between 20% and 22%. In specific examples the ratio wdv is 21.2% for dk=4.0 mm and db=2.3 mm or wdv=20.5% for dk=4.4 mm and db=2.6 mm.

The bone thread is formed as a self-tapping thread and comprises in a known manner thread flanks 6, a crest of the thread 7, a root of the thread 8 having a width B and a thread pitch P. In at least the bone thread section 5 the wall of the element 1 comprises a plurality of apertures 9 preferably having a circular cross section. The apertures 9 are arranged such that their center is located in the root of the thread 8, respectively, and the diameter of each aperture 9 is smaller than the thread pitch P and, preferably, not larger than the width B of the root of the thread such that, in the embodiment shown in FIGS. 1 and 2, the apertures 9 are located completely in the root 8 of the thread 7 and do not extend into the flanks 6. In each turn of the thread, a plurality of apertures 9 are provided in the root of the thread 8, preferably at equal distances on the helix such that, viewed in axial direction, the apertures of one turn are located preferably above the apertures of the turn lying beneath, respectively.

As can be seen from FIG. 1, the element 1 comprises, adjacent to the first end 3, a section 10 free of bone thread having an even outer wall, in which no apertures are formed. Further, in the embodiment shown, a section having an inner thread 11 is formed adjacent to the first end 3 and adjacent to the second end 4, respectively, serving for connection with the elements of an implant described hereinafter.

The tube 2 has a length that can be varied to correspond to the length of the shaft of a bone screw to be used for the particular application. The element is formed of a biocompatible material, for example, titanium or stainless steel.

The modified embodiment shown in FIG. 3 differs from the embodiment shown in FIGS. 1 and 2 in that the diameter D′ of the aperture 9′ is larger than the width B of the root of the thread 8 such that the apertures 9′ extend into the flanks 6 of the bone thread without, however, breaking through the crest 7 of the thread. In this way it is possible to form the apertures larger to achieve a better fusion with the bone. By not breaking through the crest 7 of the thread, i.e., keeping the crest 7 intact, the element in accord with the present invention avoids generating teeth having a cutting effect when screwing-in the element.

In a modification of the embodiments shown in FIGS. 1 and 2, which is shown in FIG. 8, all, or a part of the apertures 9, 9′ are provided with a countersinking 90 at the outside of the wall forming a surface roughness which facilitates growing in or growing on. The diameter of this countersinking in the direction of the screw axis is, however, smaller than the thread pitch P such that the crest 7 of the thread is intact.

In a further modification, the apertures are oval or diamond-shaped. It is decisive that they be located in the root of the thread and dimensioned such that the cutting crest of the bone thread is not damaged. In a further modification, the apertures are not provided in every turn of the thread. The apertures can have any geometry that can be provided in the wall of the tubular element.

In a further modification, the bone thread section 5 extends over the total length of the tube 2. The inner thread 11 can also extend over the total length. Alternatively, the inner thread 11 can be provided in a section at one of the ends only or may not be present at all. In case no inner thread is provided, the connection with the further parts of the implant is made for example via a snug fit.

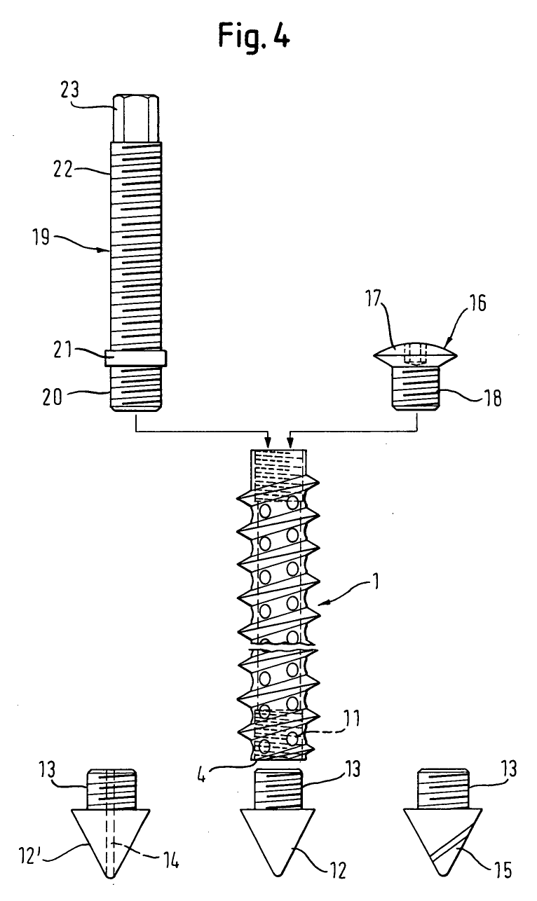

In the embodiments of implants according to the invention shown in FIGS. 4 to 6, the tubular element 1 is part of the implant, respectively. In one embodiment, the tubular element is connected with its second end 4 to a tip 12 comprising the tip conical end part and a shaft 13 having an outer thread cooperating with the inner thread 11 of the tubular element. In a modification, the tip 12′ comprises a coaxial bore passing through it which forms a channel for introducing agents at the position where the implant is to be anchored. In a further modification, the tip 15 comprises a self-tapping tip end with or without a coaxial bore passing therethrough.

In a first embodiment, the implant is formed as bone screw having a head 16 comprising (i) a head end part 17 having a slit or an inner hexagon for driving the implant and (ii) a threaded shaft 18 with an outer thread cooperating with the inner thread 11 of the tubular element 1.

In operation, a tubular element of suitable length is selected or a tube correspondingly formed is shortened to the appropriate length. Then, the tip is fixedly screwed into the tubular element 1. Thereafter, the head is screwed on the opposite end and resulting implant in the form of a bone screw can be screwed into the bone. In this case, the cavity formed by the tubular element serves for the growing-in of bone material or vessels through the apertures 9 or 9′. Alternatively, either bone material or an agent is introduced into the tubular element and the head is screwed on. Then the bone screw together with the material introduced therein is screwed into the bone.

Due to the fact that the crest of the bone thread is not damaged by the aperture but remains intact, the screwing-in takes place smoothly without a cutting effect, whereby a damage of the bone besides the damage caused by the screwing-in does not occur.

As an instrument for screwing-in of the tubular element 1 into the bone, a threaded element 19 shown in FIG. 4 is provided, which comprises a first section 20 having an outer thread cooperating with the inner thread 11 for screwing-in into the tubular element 1, a stop in form of a shoulder 21 and adjacent thereto a grip section 22 having an outer hexagon 23. The grip section 22 can be formed with or without outer thread and its length is dimensioned such that the tubular element 1 with the tip screw thereon can be positioned at a desired location in the bone. In a modification not shown the threaded element 19 comprises a coaxial channel passing therethrough, similar to tip 14.

In operation, the threaded element 19 is screwed onto the tubular element 1 and the tubular element together with the tip is immersed into the bone by means of the threaded element. Thereafter, the threaded element is screwed out, removing it from the implant.

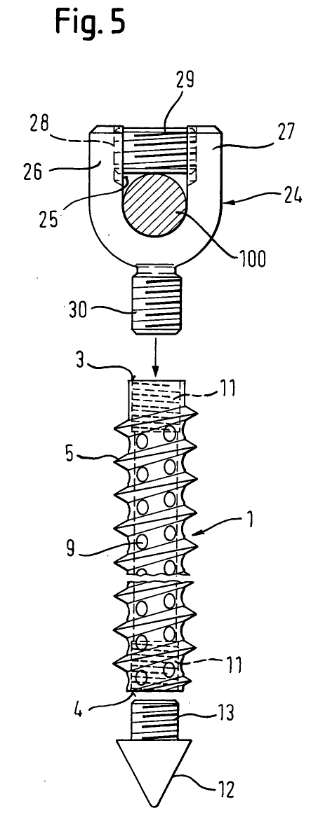

In the embodiment shown in FIG. 5 the implant consists of a screw element being formed by the tubular element 1 with tip 12 (or, alternatively tip 12′ or 15) screwed thereon, as described above, and of a receiver member 24 monoaxially connectable to the screw element for receiving a rod 100, which is provided for connecting several implants. The receiver member 24 is formed substantially cylindrically and comprises a recess 25 having a U-shaped cross section, which is dimensioned sufficiently large that the rod 100 can be placed therein and fits to the bottom of the recess. Two free legs 26, 27 are formed by the U-shaped recess 25. Adjacent to the free end, the legs 26, 27 comprise an inner thread 28 cooperating with a corresponding outer thread of an inner screw 29 to be screwed between the legs for fixation of the rod 100. On the end located opposite to the free end, the receiver member 24 comprises a threaded shaft 30 for screwing into the tubular element 1.

In operation, preferably, the implant is at first completely assembled, the tubular element being filled with agents or bone material, if necessary or desired. Thereafter, the implant is screwed into the bone. Then the implant is connected via the rod to one or several additional implants. Thereafter, the rod is fixed in the correct position by means of the inner screw. The implant is particularly suitable for the application on the spinal column. Due to the continuing crest of the bone thread of the tubular element, the vertebrae are not additionally damaged at the time of screwing-in the implant in accord with the present invention.

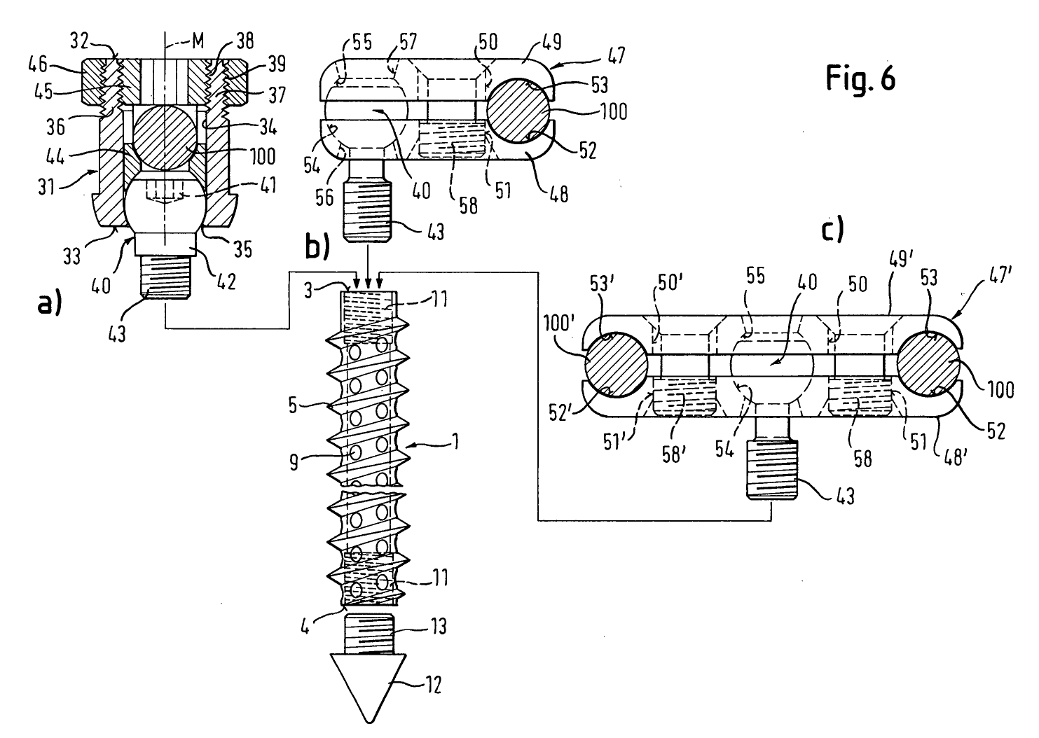

The further embodiments of implants shown in FIG. 6 are also implants which have a screw element formed of the tubular element 1 and the tip 12 (or, alternatively, tip 12′ or 15), which is connected to a receiver member for receiving a rod 100. In contrast to the embodiment according to FIG. 5, however, the connection with the rod is polyaxial.

In the embodiment shown in FIG. 6a the receiver member 31 is cylindrically shaped having a first end 32 and a second end 33 opposite to the first end. A first bore 34 extending from the first end 32 is provided which is coaxial with respect to the center axis M and which extends to a predetermined distance from the second end 33. At the second end 33 a second bore 35 is provided the diameter of which is smaller than the diameter of the first bore and which widens towards the first bore in form of a spherical segment-shaped section. The receiver member 31 comprises, beginning from the first end 32, a U-shaped recess extending perpendicular to the longitudinal axis by which the two free legs 36, 37 are formed. Adjacent to the first end 32 the legs comprise an inner thread 38. Further, an outer thread 39 is provided at the outside of the legs.

For connecting the screw element with the receiver member 31 a spherical segment-shaped screw head 40 is provided, the radius of the sphere of which is substantially equal to the radius of the hollow spherical segment-shaped section of the receiver member 31 and which is, in a loosely inserted condition, pivotable in the receiver member. The head 40 further comprises on its flattened end, which is to be directed towards the first end 32 of the receiver member 31, a recess 41 for engagement with a screw driver. At the opposite end, the screw head 40 comprises a cylindrical neck 42 having an outer diameter corresponding to the outer diameter of the tubular element 1. From the neck a projection 43 extends which has an outer thread for screwing the screw head 40 into the tubular element.

For fixation of the head 40 in the receiver member and for fixation of the angular position of the screw element in this way, a pressure element 44 is provided which is cylindrically shaped such that it is slidable in the receiver member, and which comprises at its one end a spherical recess for receiving a section of the screw head and at the opposite end a U-shaped recess for receiving the rod. The pressure element 44 comprises a coaxial bore to allow screwing-in of the screw element when the pressure element is inserted. For fixation of the head 40 and the rod 100 an inner screw 45 is provided which can be screwed between the legs 36, 37. For securing of the fixation, a securing nut 46 is provided which can be screwed on the receiver member 31.

In operation, first the screw element consisting of the tip 12, the tubular element 1 which is filled, if applicable, with bone material or an agent, and the screw head is assembled. Thereafter, the screw element and the pressure element are inserted into the receiver member and the screw element is screwed into the bone or the vertebra. The connection with other implants via the rod is made in a known manner.

The fixation of the head and the rod is not limited to the embodiment described but every fixation of the head and the receiver member of known polyaxial screws can be used.

In the embodiment shown in FIGS. 6b) and 6c) the polyaxial connection with the rod 100 is not made in direction of the screw axis as in the example according to FIG. 6a), but laterally shifted with respect to the screw axis.

The implant according to FIG. 6b) comprises a screw element consisting of a tubular element 1, the tip 12 and the spherical segment-shaped head 40 shown in FIG. 6a), and a two-part holder 47 accommodating the head 40 with a lower part 48 facing the tubular element 1 and an upper part 49 opposite to the tubular element 1, which together encompass the rod 100. The upper part 49 and the lower part 48 are formed identically and are arranged mirror-inverted with respect to each other. They comprise a central bore 50, 51 being provided with an inner thread and which has on the surface opposite to the other part 48, 49, respectively, a countersinking. At a distance laterally from the bore 50, 51, a recess 52, 53 for supporting the rod is provided which is formed cylindrical segment-shaped towards the respective other part 48, 49. At the other side of the bore 50, the lower part 48 and the upper part 49 comprise at the side facing the respective other part a spherical segment-shaped recess 54, 55 for supporting the screw head 40. Adjacent to the surface opposite to the other part 48, 49, coaxial with the recess 54, 55, there is a recess 56, 57 widening towards the outside.

Lower part 48 and upper part 49 of the holder are connected by a screw 58 which can be inserted into the inner thread of the upper part and which can be screwed into the inner thread of the lower part. In the part which is guided through the upper part 49, the screw has a diameter which is smaller than the diameter of the inner thread of the upper part and in its part guided through the lower part, the screw comprises an outer thread cooperating with the inner thread of the lower part. The cylindrical segment-shaped recesses 52, 53 and the spherical segment-shaped recesses 54, 55 are dimensioned such and arranged such with respect to each other that in a state in which the rod and the head are supported, the lower part 48 and the upper part 49 are aligned and parallel and have a distance from each other.

In operation, first, the screw element is assembled. The upper part and the lower part of the holder are rotated by an angle of 90° with respect to each other by loosening of the screw 58 such that the screw element can be inserted into the lower part 48. The screw element is inserted until its head 40 is adjacent to the spherical segment-shaped recess 54. Thereafter, the screw element is screwed into the bone. Then the rod 100 is accommodated and the upper part 49 is rotated by an angle of 90°. After adjustment of the angular position of the screw head 40 in the holder and of the position of the rod a fixation is carried out by tightening the screw 58.

The implant is suitable in particular for the fixation of fractures of the pelvis and of long bones.

The embodiment shown in FIG. 6c) differs from the embodiment represented in FIG. 6b) in that the holder 47′ for holding of two rods 100, 100′ comprises a lower part 48′ and an upper part 49′, which are symmetrically formed in themselves, respectively.

The lower part 48′ and the upper part 49′ are formed symmetrically with respect to a plane defined by the center line of the rods 100, 100′ and the center of the spherical segment-shaped head 40 of the screw and comprise two bores 50, 50′, 51, 51′, two cylindrical segment-shaped recesses 52, 52′, 53, 53′, respectively. Two fixation screws 58, 58′ are provided for fixation. The operation corresponds to that of the embodiment described above with the only difference that two rods have to be fixed.

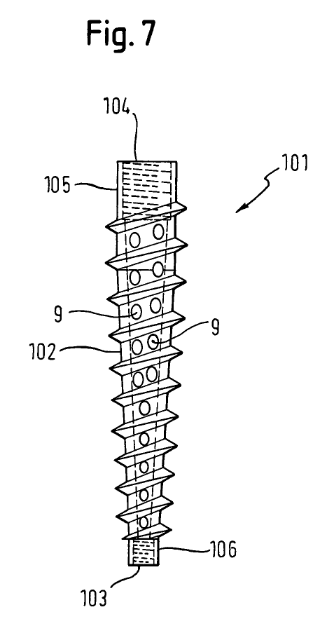

In an embodiment shown in FIG. 7 the tubular element 101 is not formed cylindrically entirely but comprises a middle conical bone thread section 102 tapering towards the end 103 of the element which is to be connected to the tip. Adjacent to the conical section a cylindrical section 105, 106 extends on both sides of the conical section up to the opposite ends 103, 104, respectively, which comprises an inner thread for connecting to the tip at one end or to a head, a threaded element or a receiver member as described before at the other end.

In a modification, the cylindrical section 106 to be connected with the tip is not provided but the free end of the conical bone thread section acts itself as the tip.

The invention has been described in detail. However, it will be appreciated that, upon consideration of the specification and drawings, those skilled in the art may make modifications and improvements within the spirit and scope of the claims.

What is claimed is:

1. A tubular element useful for an implant for spine or bone surgery, the tubular element comprising two ends and a wall having a bone thread section with (i) a bone thread comprising thread flanks, a crest and a root, and (ii) a plurality of apertures in the wall in the bone thread section, wherein at least one end of the element is structured and arranged to receive a head or a tip, and wherein the apertures are located in the root of the bone thread and comprise dimensions such that the crest of the bone thread is intact.

2. The tubular element according to claim 1, wherein at least one aperture comprises dimensions such that a largest linear distance across the aperture is smaller than the distance between adjacent thread flanks at the root of the thread.

3. The tubular element according to claim 1, wherein at least one aperture comprises dimensions such that a largest linear distance across the aperture is larger than the distance between adjacent thread flanks at the root of the thread, wherein the aperture then extends into at least one of the adjacent thread flanks.

4. The tubular element according to claim 1, wherein at least one of the apertures comprises an exterior countersink area.

5. The tubular element according to claim 1, wherein a plurality of equally distanced apertures are provided in each turn of the thread.

6. The tubular element according to claim 1, wherein at least one end comprises a section having an inner thread for receiving a head component.

7. The tubular element according to claim 1, wherein the bone thread section comprises a conical shape.

8. An implant comprising a tubular element having two ends and a tip detachably connected at one end of the tubular element, wherein the tubular element further comprises a wall having a bone thread section with (i) a bone thread comprising thread flanks, a crest and a root, and (ii) a plurality of apertures in the wall in the bone thread section, wherein at least one end of the element is structured and arranged to receive a head or a tip, and wherein the apertures are located in the root of the bone thread and comprise dimensions such that the crest (7) of the bone thread is intact.

9. The implant according to claim 8, further comprising a head that is detachably connected to the other end of the tubular element.

10. The implant according to claim 9, wherein the head comprises a screw head.

11. The implant according to claim 9, wherein the head comprises a cylindrical element having an exterior thread.

12. The implant according to claim 9, wherein the head comprises a receiver member for holding a rod.

13. The implant according to claim 12, wherein the head and the tubular element are connected monoaxially with each other.

14. The implant according to claim 12, wherein the head and the tubular element are connected polyaxially with each other.

15. A method for inserting an implant into bone tissue, the method comprising:
providing a tubular element of suitable length, the tubular element comprising two ends and a wall having a bone thread section with (i) a bone thread comprising thread flanks, a crest and a root, and (ii) a plurality of apertures in the wall in the bone thread section, wherein at least one end of the element is structured and arranged to receive a head or a tip, and wherein the apertures are located in the root of the bone thread and comprise dimensions such that the crest of the bone thread is intact;
fixing a tip portion onto one end of the tubular element;
fixing a head portion onto the other end of the tubular element, thereby forming a bone screw; and
inserting the bone screw into the bone.

16. The method of claim 15, further comprising introducing bone material or an agent into the tubular element before fixing the head portion.

17. The method of claim 15, wherein the head portion comprises a receiver member for receiving a rod, the method further comprising fixing the rod in the receiver member.

18. The method of claim 17, further comprising introducing bone material or an agent into the tubular element before fixing the head portion.

19. A method for fixing bone fractures, the method comprising:
providing a tubular element of suitable length, the tubular element comprising two ends and a wall having a bone thread section with (i) a bone thread comprising thread flanks, a crest and a root, and (ii) a plurality of apertures in the wall in the bone thread section, wherein at least one end of the element is structured and arranged to receive a head or a tip, and wherein the apertures are located in the root of the bone thread and comprise dimensions such that the crest of the bone thread is intact;
fixing a tip portion onto one end of the tubular element;
introducing bone material or an agent into the tubular element;
fixing a head portion onto the other end of the tubular element, thereby forming a bone screw;
inserting the bone screw into the bone to be fixed; and
allowing bone tissue to grow around the bone screw and into the apertures.

20. A method for stabilizing the spine, the method comprising:
providing a tubular element of suitable length, the tubular element comprising two ends and a wall having a bone thread section with (i) a bone thread comprising thread flanks, a crest and a root, and (ii) a plurality of apertures in the wall in the bone thread section, wherein at least one end of the element is structured and arranged to receive a head or a tip, and wherein the apertures are located in the root of the bone thread and comprise dimensions such that the crest of the bone thread is intact;
fixing a tip portion onto one end of the tubular element;
introducing bone material or an agent into the tubular element;
fixing a head portion onto the other end of the tubular element, wherein the head portion comprises a receiver member for receiving a rod, thereby forming a bone screw with a receiver member;
inserting the bone screw into the spine;
fixing the rod in the receiver member; and
allowing bone tissue to grow around the bone screw and into the apertures.

21. The method of claim 20, further comprising:
providing one or more additional tubular elements, each additional element comprising two ends and a wall having a bone thread section with (i) a bone thread comprising thread flanks, a crest and a root, and (ii) a plurality of apertures in the wall in the bone thread section, wherein at least one end of the element is structured and arranged to receive a head or a tip, and wherein the apertures are located in the root of the bone thread and comprise dimensions such that the crest of the bone thread is intact;
fixing a tip portion onto one end of each additional tubular element;
introducing bone material or an agent into one or more of each additional tubular element;
fixing a head portion onto the other end of each additional tubular element, wherein the head portion comprises a receiver member for receiving a rod, thereby forming an additional bone screw with a receiver member;
inserting each additional bone screw into a separate location of the spine;
fixing the rod into the receiver member of each additional bone screw; and
allowing bone tissue to grow around each additional bone screw and into the apertures.
